(19) United States
(12) Patent Application Publication
(10) Pub. No.: US 2022/0183564 A1
(43) Pub. Date: Jun. 16, 2022

(54) PHOTOACOUSTIC BREAST IMAGING SYSTEM AND METHOD

(71) Applicant: The Research Foundation for University of New York, Buffalo, NY (US)

(72) Inventors: Jun XIA, Amherst, NY (US); Nikhila NYAYAPATHI, Buffalo, NY (US); Huijuan ZHANG, Buffalo, NY (US)

(21) Appl. No.: 17/600,085

(22) PCT Filed: Mar. 30, 2020
(86) PCT No.: PCT/US20/25848

§ 371 Date: Sep. 29, 2021

Related U.S. Application Data
(60) Provisional application No. 62/826,979, filed on Mar. 29, 2019.

(51) Int. Cl. A61B 5/00

(52) U.S. Cl. CPC A61B 5/0095; A61B 5/4312; A61B 5/708

(57) ABSTRACT

Systems and methods are provided for imaging a sample. The system may include a pulsed light source configured to irradiate a region of interest in a sample from a first side and a second side opposite the first side; a first ultrasound transducer configured to receive acoustic waves induced at the region of interest and received from the first side of the sample; a second ultrasound transducer configured to receive acoustic waves induced at the region of interest and received from the second side of the sample; and a controller.

### CROSS-REFERENCE TO RELATED APPLICATIONS

This application claims priority to U.S. Provisional Application No. 62/826,979, filed on Mar. 27, 2019, now pending, the disclosure of which is incorporated herein by reference.

### FIELD OF THE DISCLOSURE

The present disclosure relates to breast imaging, and in particular to photoacoustic breast imaging.

### BACKGROUND OF THE DISCLOSURE

Breast cancer is one of the most common cancers—one in eight women will have the diagnosis during their lifetime in the United States. It is the second leading cause of cancer-related deaths. With the modern standard of care, early breast cancers detected by screening have achieved more than 99% five-year survival. However, existing techniques to visualize and diagnose breast cancer have certain limitations. Although mammogram is the most often performed standard procedure for screening, it has decreased sensitivity in women with dense breasts, along with exposure to ionizing radiation. While Magnetic Resonance Imaging (MRI) has high sensitivity and specificity in dense-breast patients, it is very expensive and requires the use of intravenous contrast agents which can cause an allergic reaction, kidney damage, and permanent deposition in the central nervous system. Another popular imaging technique is ultrasound. Ultrasound is portable, safe, and patient friendly because it does not require strong compression of the breast, as is the case with mammogram, or require the patient to be confined within a narrow space, as is the case with MRI. However, ultrasound technology exhibits variability between operators and takes up considerable physician time, therefore it is commonly used as an adjunctive modality to complement mammogram. An automated breast ultrasound system has been developed (Invenia ABUS) to eliminate operator error; however, it only looks at acoustic contrasts and is known to increase the rate of false positives when used as an aid in the screening process. Also, the supine geometry in the ABUS system requires additional training for radiologists to interpret and correlate data with mammograms, which is the most widely used imaging modality of choice for breast screening procedures. While Diffuse Optical Tomography (DOT) has also been proposed in recent studies, it has a poor spatial resolution, due to optical scattering, and cannot visualize the biological tissue clearly. Therefore, there is a need for a new, safe, and accurate imaging modality that can overcome the aforementioned limitations and be used in a wide range of patient populations.

### BRIEF SUMMARY OF THE DISCLOSURE

Photoacoustic systems and methods with high spatial resolution, fast imaging capability, and convenient correlation is presented. The system can be used with all imaging modalities used for breast cancer detection—mammography, MRI, and ultrasound. The upright positioning allows for easy and quick patient imaging. Having light illumination from both sides of the breast stems the exponential decay of signal inside tissue to an extent, making it possible to get better images. Acquiring photoacoustic data from two transducers and combining it bolsters the signal intensity at the deepest portion of the breast where there is maximum attenuation. This improves the ability to image deep into the breast without missing vascular features. After reconstruction and combination of images from top and bottom, an exemplary embodiment could image completely through 7 cm of breast tissue. With high resolution demonstrated by the DSM using the 3D focal-line reconstruction, we have the capability to detect small tumors in the sub-millimeter range, provided they exhibit sufficiently developed angiogenesis. Since the DSM acquires ultrasound simultaneously, it can obtain a comprehensive analysis of the breast by overlaying PA images on ultrasound.

### DESCRIPTION OF THE DRAWINGS

For a fuller understanding of the nature and objects of the disclosure, reference should be made to the following detailed description taken in conjunction with the accompanying drawings, in which:

FIG. 1: A diagram of a system for imaging a sample according to an embodiment of the present disclosure.

FIG. 2: An exemplary pulse/response order for use with two modalities: photoacoustic and ultrasound.

FIG. 3: An exemplary embodiment of a Dual Scan Mammoscope (DSM) according to the present disclosure. (a) A 3D schematic drawing of the exemplary system.

FIG. 4: A cross-sectional view of a system according to another embodiment of the present disclosure, highlighting the light illumination and acoustic detection paths.

FIG. 5: Photoacoustic amplitude for three different ultrasound coupling media available in the market. (a) Photograph of three different ultrasound gels in agar wells. (b) Maximum amplitude projection (MAP) showing photoacoustic absorption of Polysonic® ultrasound lotion at 1064 nm.

FIG. 6: Quantification of lateral and elevation resolutions of a DSM system. The imaging targets are human hairs embedded in an agar gel. (a) Images of hairs placed along the lateral direction of the transducer array. These images were used to quantify the elevation resolution of the system. (b) Images of hairs placed along the elevation direction of the transducer array. These images were used to quantify the lateral resolution of the exemplary system. The top row are images reconstructed using the 2D reconstruction algorithm. The bottom row contains images reconstructed using the 3D focal-line reconstruction algorithm.

FIG. 7: Photoacoustic image of a volunteer with breast size D, scattered fibroglandular breast density, and thickness of 70 mm after compression. (a) A schematic drawing of craniocaudal (CC) view image orientation in an X-ray Mammogram. (b) Depth-encoded maximum intensity projection image of the breast, formed by the 2D reconstruction algorithm (with a limited number of color-coded vessels labeled for illustration). (c) Depth-encoded maximum intensity projection image of the breast, formed by the 3D focal-line reconstruction algorithm (with a limited number of color-coded vessels labeled for illustration). The Roman numbers mark the same vessels shown in (b), (c), and FIG. 9.

FIG. 8: Comparison of Photoacoustic data with contrast-enhanced MRI results of the volunteer. (a) MAP of contrast-enhanced MRI subtraction images for right and left breasts. Red box indicates region of breast imaged by DSM. (b) Photoacoustic images of the same pair of breasts. The numerals point to the same vessels in MRI and PA data.

FIG. 9: A three-dimensional representation of ultrasound images overlaid with photoacoustic images of a volunteer with breast size D. (a & b) sagittal view. (c & d) axial view. (e & f) coronal view. The frame number associated with each view represents the corresponding frame which the slice was extracted. The Roman numbers mark the same vessels shown in FIGS. 7(b) and 7(c).

FIG. 10: UV-Vis spectroscopy results for three ultrasound gels tested. From left to right are the absorption spectra for the Aquasonic Clear®, Polysonic®, and Scan® gels. Because the Polysonic® lotion is a white milky consistency, the spectrum is mostly contributed by scattering instead of absorption. Although the Aquasonic® Clear gel has the least amount of absorption, the Scan® gel has comparable absorption in the NIR-II window which was a focus of an experimental embodiment. Among the three, the Scan® gel was the least viscous and it was very easy to remove any air bubbles.

FIG. 11: Overview of how an imaging region from each transducer was chosen for an exemplary system.

FIG. 12: Photoacoustic images of tubes filled with India ink placed in an agar block. (a) Image acquired by the bottom transducer, (b) image acquired by the top transducer. (c) Adding top and bottom data without alignment. (d) Adding top and bottom data after cross-correlation-based alignment. Data from both top and bottom transducers were combined to represent the complete phantom. The offset was calculated using the cross-correlation technique, and verified by manually calculating the offset for similar looking features.

FIG. 13: Tissue mimicking agar phantom with 1% Intralipid 20% (IL-20). (a) A photo of four black hair strands placed 2 cm apart. (b) Photo of the hair strands from (a) immersed in agar gel.

FIG. 14: Photoacoustic image of a 65-year-old volunteer with breast size B, scattered fibroglandular breast density and thickness of 30 mm after compression. Depth-encoded maximum intensity projection image of the breast, formed by an exemplary 2D reconstruction algorithm.

FIG. 15: Photoacoustic image of a healthy volunteer with breast size DD and thickness of 55 mm after compression. (a) Depth-encoded maximum intensity projection image of the breast, formed by the 2D reconstruction algorithm. (b) Depth-encoded maximum intensity projection image of the breast, formed by an exemplary 3D focal-line reconstruction algorithm.

FIG. 16: A three-dimensional representation of ultrasound (gray) images overlaid with photoacoustic images (color) of a volunteer with breast size B. (a & b) sagittal view. (c & d) axial view. (e & f) coronal view. The frame number associated with each view represents the corresponding frame which the slice was extracted.

FIG. 17: A three-dimensional representation of ultrasound (gray) images overlaid with photoacoustic images (color) of a volunteer with breast size DD. (a & b) sagittal view. (c & d) axial view. (e & f) coronal view. The frame number associated with each view represents the corresponding frame which the slice was extracted.

FIG. 18: Maximum depth depicted from one transducer is 35 mm.

### DETAILED DESCRIPTION OF THE DISCLOSURE

The photoacoustic (PA) effect was first discovered by Alexander Graham Bell in 1880 when he observed that sound was generated due to absorption of modulated sunlight. The field did not see major advancements until the mid-2000s when high-power pulsed lasers became available for photoacoustic excitation. This paved the way for rapid improvements in instrumentation, image reconstruction techniques, molecular imaging, and applications in biological research. Photoacoustic signals are generated when a short, pulsed laser light irradiates a biological tissue sample, causing thermoelastic expansion, which generates acoustic waves. These photoacoustic pressure waves are then detected by an ultrasound transducer array. Inversing the signal detection process allows us to get an image of optical absorption. For deep-tissue imaging, near infrared (NIR) wavelengths are advantageous due to their weak optical absorption and scattering. Hemoglobin, which is a main absorber in the NIR region, allows for label-free photoacoustic imaging of vasculatures in breast tissue.

Photoacoustic imaging can potentially overcome major limitations that current breast imaging techniques possess. Firstly, the technique uses diffused laser light for illumination, making it completely free of ionizing radiation, as opposed to an X-ray mammogram. Secondly, since this modality is based on ultrasound, which is weakly scattered in biological tissue, the spatial resolution of photoacoustic tomography (PAT) is comparable to ultrasound (<1 mm) and is much better than that of pure optical tomography techniques such as Diffuse Optical Tomography (DOT). Additionally, unlike contrast-enhanced MRI, PAT uses hemoglobin as endogenous contrast and is therefore completely label-free and non-invasive. The extent of angiogenesis and presence of microvasculature—which are biomarkers for malignancy—can potentially be determined from PA signal intensity. These features can help differentiate benign masses from those that require a biopsy, preventing unnecessary trauma for the patient.

The present disclosure may be embodied as a system for scanning a sample. In some embodiments, the system is a Dual Scan Mammoscope (DSM) that represents a significant advancement in PAT. A test embodiment of the DSM achieved an imaging depth of 7 cm, which has never before been reported by any other PAT breast imaging system. The improved performance was achieved while imaging the patient standing upright, with the breast compressed and scanned along the craniocaudal (CC) plane. To get a complete visualization of the imaging region, two linear array transducers are employed in combination with co-planar-illuminated linear output fibers to scan from both top and bottom of the breast. This design ensures improved light delivery and acoustic detection, enabling deeper imaging depth. It is noted that embodiments of the present disclosure may be used along other planes and with patients in other positions (seated, prone, etc.) and are within the scope of the disclosure.

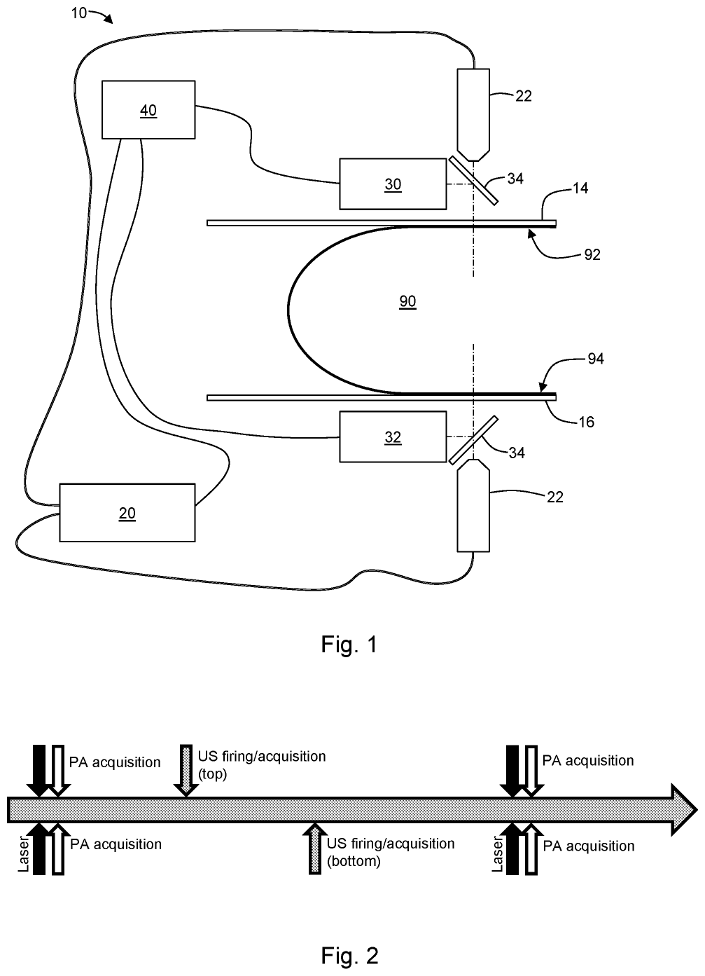

With reference to FIG. 1, in a first aspect, the present disclosure may be embodied as a system 10 for imaging a sample. The system 10 includes a pulsed light source 20. configured to irradiate a region of interest in the sample 90 from a first side 92 and a second side 94 opposite the first side. The light source 20 may be configured to emit light at any frequency/wavelength selected to induce acoustic waves in the sample. For example, the light source may be configured to emit light in at least a portion of the visible region, at least a portion of the infrared region, and/or at least a portion of the near-infrared (NIR) region. The light source may be, for example, a laser, such as an Nd:YAG laser. The system 10 may further comprise fiber optic bundles 22 configured to receive light from the light source 20 and transmit the received light to the first side of the sample and the second side of the sample. The system may include a first sample surface 14 and a second sample surface 16 configured such that the sample is held between the first and second sample surfaces.

The system includes a first ultrasound transducer 30 configured to receive acoustic waves from the region of interest (e.g., induced in the region of interest) and received from the first side of the sample. A second ultrasound transducer 32 is configured to receive acoustic waves induced at the region of interest and received from the second side of the sample. The first and/or second ultrasound transducers may be linear arrays. The transducers may be configured for acoustic waves of any suitable frequency or frequencies. For example, the transducers may be configured for use with acoustic waves having frequencies of from 1 MHz to 100 MHz. In some embodiments, a first mirror 34 is configured to cause light from the light source at the first side to be coincident with an acoustic wave received at the first ultrasound transducer 30 (e.g., along a primary transmit or receive axis of the transducer, etc.) Similarly, a second mirror 36 may be configured to cause light from the light source at the second side to be coincident with an acoustic wave received at the second ultrasound transducer 32 (e.g., along a primary transmit or receive axis of the transducer, etc.) The first mirror and/or the second mirror may be any suitable mirrors, such as, for example, a dichroic mirror, an acoustic mirror, etc. On each of the first side and the second side, the source of light and the transducer may be configured in any various configurations. For example, FIG. 1 depicts the light and acoustic signals at 90° to one another (and the mirror at)45°. In some embodiments, the component (light and transducer) positions may be swapped from the arrangement shown in FIG. 1. In other embodiments, the components are at angles other than 90° with respect to each other, for example, angles from 0° to 180° and beyond 180°.

The system 10 includes a controller 40 in electronic communication with the light source, the first ultrasound transducer, and the second ultrasound transducer. The controller may be, for example, a microprocessor, a computer, a field-effect gate array (FPGA), an application-specific integrated circuit (ASIC), discrete logic, or multiples and combinations of these or other such controllers, as is known in the art. The controller is configured to trigger a first pulse from the light source and receive a first acoustic wave signal from the first ultrasound transducer, the first acoustic wave signal corresponding to the first pulse (i.e., an acoustic wave induced in the region of interest by the first pulse and received on the first side). The controller is further configured to receive a second acoustic wave signal from the second ultrasound transducer. The second acoustic wave signal corresponds to the first pulse (i.e., an acoustic wave induced in the region of interest by the first pulse and received on the first side). The controller is further configured to construct an image of the region of interest based on the received first acoustic wave signal and the second acoustic wave signal.

The system may further include a scan stage configured to re-direct the light in a scan direction so as to impinge on a second region of interest of the sample. Similarly, the scan stage translates the first ultrasound transducer and the second ultrasound transducers so as to receive acoustic waves from the second region of interest. The controller may be further configured to actuate the scan stage to move the scan stage to the second region of interest and repeat the trigger, receive, and construct steps (described above) at the second region of interest. The constructed images at the region of interest and second region of interest may be joined. In this way, the controller may be configured to continue repeating the translate, trigger, and receive steps so as to construct an image (e.g., joined images) of the sample over a distance in the scan direction. In some embodiments, the image is constructed based on first acoustic wave signals, and the adjoining image is constructed by matching the second acoustic wave signals to the first acoustic wave signals (e.g., to match an offset of the first and second ultrasound transducers).

In some embodiments, the controller 40 is further configured to trigger a first acoustic pulse from the first ultrasound transducer and receive a first echo signal from the first ultrasound transducer. The controller may be configured to trigger a second acoustic pulse from the second ultrasound transducer and receive a second echo signal from the second ultrasound transducer. The controller may be configured to trigger the second acoustic pulse so that it does not interfere with the first acoustic pulse and the first echo signal. In this way, the system may be used in photoacoustic and/or pulse-echo ultrasound. An exemplary pulse order for dual-mode capture is depicted in FIG. 2. In some embodiments, the controller is further configured to perform ultrasound tomography. For example, the first acoustic pulse and/or the second acoustic pulse may be configured for tomographic reconstruction as is known in the art.

The system may further include a first vessel configured to hold a medium (for example, de-ionized water, etc.) on the first side of the sample. The first vessel has a first sample surface configured to be coupled to the first side of the sample. In this way, the first ultrasound transducer may be contained within the first vessel (e.g., at least partially disposed in the medium) such that acoustic waves from the sample are received by transmission through the first sample surface. The system may further include a second vessel configured to hold a medium (for example, de-ionized water, etc.) on the second side of the sample. The second vessel has a second sample surface configured to be coupled to the second side of the sample. In this way, the second ultrasound transducer may be contained within the second vessel (e.g., at least partially disposed in the medium) such that acoustic waves from the sample are received by transmission through the second sample surface.

The first and second sample surfaces may be made from a material that is substantially transparent to acoustic waves (for example, ultrasound frequencies, ranging from a few MHz (or less) to 100 MHz (or more)) and light (for example, visible or infrared regions, for example, near-infrared (NIR)). For example, the first and second sample surfaces may be made from a material that transmits greater than 70%, 75%, 80%, 85%, 90%, or 95% of light (or any percentage value between such values) at the light source wavelength (for example, visible or infrared regions, for example, near-infrared (NIR)). The first and second sample surfaces may be made from a material that transmits greater than 70%, 75%, 80%, 85%, 90%, or 95% of acoustic signal (or any percentage value between such values) at the ultrasound frequencies (for example, ranging from a few MHz (or less) to 100 MHz (or more)). In a particular example, the first and second sample surfaces are made from a fluorinated ethylene propylene (FEP) plastic film having a thickness of 50 μm or less.

The first vessel or the second vessel may be mounted to a translatable stage configured to translate the first and second vessels nearer to, or further from, each other, thereby compressing or uncompressing the sample, respectively. In this way, the sample may be compressed before “scanning” (e.g., the triggering and receiving steps). In some embodiments, the first and second vessels are each mounted to respective translatable stages.

In another aspect, the present disclosure may be embodied as a method for imaging a sample. The method includes triggering a first light pulse from a light source configured to irradiate a region of interest in the sample from a first side of the sample and/or a second side of the sample opposite the first side; receiving, from the first side of the sample, a first acoustic wave signal induced at the region of interest by the first pulse; receiving, from the second side of the sample, a second acoustic wave signal induced at the region of interest by the first pulse; and constructing an image of the region of interest based on the received first acoustic wave signal and second acoustic wave signal. The steps of the method (e.g., the steps of triggering, receiving, and constructing) may be repeated at a second region of interest of the compressed sample. In this way, the method may be used to scan over an area (e.g., image a volume) of the sample.

The method may further include triggering a first acoustic pulse configured to impinge upon the region of interest from the first side of the sample; and receiving, from the first side of the sample, a first echo signal induced by the first acoustic pulse. In some embodiments, the method further includes triggering a second acoustic pulse configured to impinge upon the region of interest from the second side of the sample, and wherein the second acoustic pulse is timed so as not to interfere with the first acoustic pulse and first echo signal; and receiving, from the second side of the sample, a second echo signal induced by the second acoustic pulse.

22. The method of claim 18, further comprising repeating the steps of translating, triggering, and receiving so as to construct an image of the sample over a distance in the scan direction.

23. The method of claim 18, further comprising compressing at least a portion of the sample, wherein the sample is compressed along an axis from the first side to the second side.

### Methods

### A. System Design

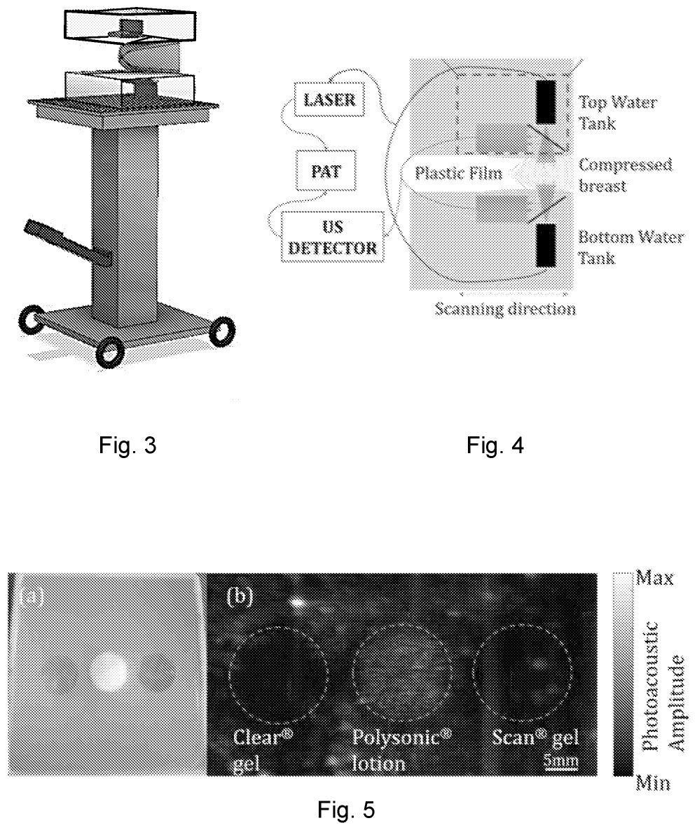

An exemplary embodiment of the present system was built on a foot-operated mobile lift table with adjustable height. An optical breadboard was clamped to the lift table. One of the water tanks (bottom) was fixed to the breadboard. Another water tank (top) was mounted on rods with clamps, that can climb up and down to allow for compression depending on breast sizes. Both tanks are made with clear acrylic sheets. To ensure light and acoustic transmission, the top tank has an opening bottom sealed with fluorinated ethylene propylene (FEP) plastic film (McMASTER-Carr), while the bottom tank has an opening top sealed with another FEP plastic film. Through pulse-echo measurements, it was verified that a film thickness of 50 μm had no acoustic attenuation at 5 MHz. The effect of the film on light propagation was also verified and it was found that the light transmission to be greater than 97%. For linear scanning of both the transducer and the optical fiber output along the length of the breast, we utilized a 20 cm stroke translation stage mounted on the breadboard (FIG. 3). Both ultrasound transducers were 128-element linear arrays with curved elements to facilitate acoustic focusing without using a lens. The element width was 8.6 cm and the central frequency is 2.25 MHz (custom made by Imasonics, Inc.). The light source used in the experimental embodiment was a 10 ns Nd:YAG laser with 10 Hz pulse repetition rate, at 1064 nm wavelength output (Continuum, SL III). It was coupled to a bifurcated line output fiber with input diameter of 1 cm and output length of 9 cm (Dolan-Jenner Industries). A portable Q-switched Nd:YAG laser with pulse repetition rate of 6 Hz and 1064 nm output wavelength was also used for clinical imaging procedures. Synchronization of light delivery and acoustic detection was achieved with trigger output from the laser. To achieve co-planar light illumination and acoustic detection which allows for optimal photoacoustic detection, a 3D printed holder was designed such that a dichroic mirror (TECHSPEC® cold mirror, Edmund Optics Inc.) could be attached at a 45-degree angle to the transducer. The dichroic mirror allows for 97% of 1064 nm light to pass through when incident at a 45-degree angle. Both top and bottom transducers were similarly fixed in a 3D printed holder with each branch of the bifurcated fiber bundle. Both transducers were aligned such that there is maximum overlap in their imaging regions. During imaging, the two transducer-fiber bundle sets were immersed into the corresponding water tanks as shown in FIG. 4. 1064 nm light was used as it can penetrate deeper into the tissue with lesser scattering and it is fundamental wavelength for Nd:YAG laser. Energy irradiated on the living body was measured to be 21 mJ/cm², which is well below the ANSI safety limit of 100 mJ/cm² for 1064 nm light. Software code was developed using Matlab and a 256-channel DAQ (Verasonics, Inc.) such that the ultrasound and photoacoustic acquisitions were interleaved, with ultrasound being acquired in between two consecutive laser pulses. To ensure high speed imaging, we used plane wave ultrasound excitation where all transducer elements are excited at the same time. After excitation, all elements acquire the signals simultaneously. To avoid interference, this process alternates between the two transducers, top and bottom, at an interval of 24 msec. With this setup, we have an imaging window of 10 cm×8.6 cm, with thickness and scan length determined by the breast volume being imaged.

### B. Breast Coupling

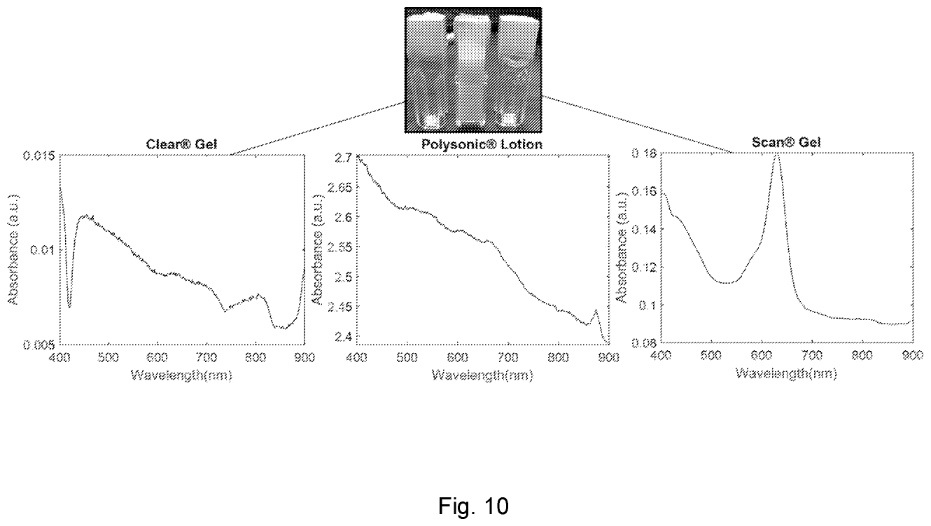

One of the challenges we faced was the removal of air-bubbles formed after ultrasound gel is applied to the breast surface. Due to the viscous nature of the regular clear ultrasound gel (AquaSonic Clear®, Parker Laboratories, Inc.) commonly used in clinics, we found that air-bubbles were trapped between the skin surface and plastic film of the water tank. Removal of these bubbles became a challenge, especially for the bottom water tank, as once the breast was placed, there is no room to maneuver for the operator. To resolve this issue, we tested the photoacoustic absorption at 1064 nm for two of the less viscous ultrasound gels found in the market, Scan® gel and Polysonic® ultrasound lotion, against the standard Aquasonic Clear® gel, all from Parker Laboratories, Inc. We placed the three gels in agar wells as shown in FIG. 5(a), and scanned the sample using 1064 nm wavelength. From FIG. 5(b), we can see there is no absorption from the Scan® gel and Aquasonic Clear® gel at this wavelength, whereas the Polysonic® ultrasound lotion has absorption at 1064 nm. We also measured the absorbance of the gels using UV-Vis spectroscopy (FIG. 10) and found that the Scan® gel has negligible absorbance in the 1064 nm range. Therefore, we surmised that the less viscous Scan® ultrasound gel, which has no absorption at 1064 nm is the ideal solution to our problem. All our following experiments were performed using the Scan® gel. During human experiments, we observed that with the Scan® gel, air-bubbles were minimal.

### C. Imaging Procedure

The experimental DSM was configured with an upright geometry where the breast was placed between two water tanks and mildly compressed using plastic films in the craniocaudal plane. Both water tanks were filled with deionized water (DI water) to minimize air-bubbles. The bottom water tank was fixed on the breadboard and the lift-table was adjusted such that the patient could stand comfortably. We first put ultrasound gel on the plastic film of the bottom tank and placed the breast on it. Then, we applied ultrasound gel to the top surface of the breast and the top water tank is gradually rolled down with the help of two rod clamps mounted on optical rods with teeth. The top water tank was lowered until desired breast compression was achieved. After making sure that both transducers were positioned correctly, the breast was scanned along its length with a step size of 0.1 mm/laser pulse. We determined the scanning time based on the length of breast tissue that had been compressed craniocaudally. For example, the scan time for a 6 cm breast was 60 seconds with our calibrated 0.1 mm/pulse step size. After a first breast, we cleaned the plastic films with wipes to remove excess gel and imaged the other breast in a similar fashion. There was no need to replace water as it did not come in contact with the patient. Therefore, the imaging procedure was very fast. The actual scanning time for one breast was 1 minute and the complete imaging session, including patient preparation took about 15 minutes. With mild compression, the patient did not feel any pain during the imaging procedure.

### D. Reconstruction and Alignment

For reconstruction, we used the universal back-projection algorithm for 2D reconstruction and the focal-line-based algorithm for 3D reconstruction. The 2D approach reconstructs each imaging plane individually and then stack multiple planes to get a 3D image. For 3D reconstruction, we computed the time-of-arrival in 3D using the focal-line concept and each imaging plane was reconstructed using the 3D raw-channel data. This approach allows us to get a better spatial resolution along the scanning direction. To display the 3D image, we used depth-encoded maximum intensity project (MIP), in which blue represents shallow depth and red represents deepest structures.

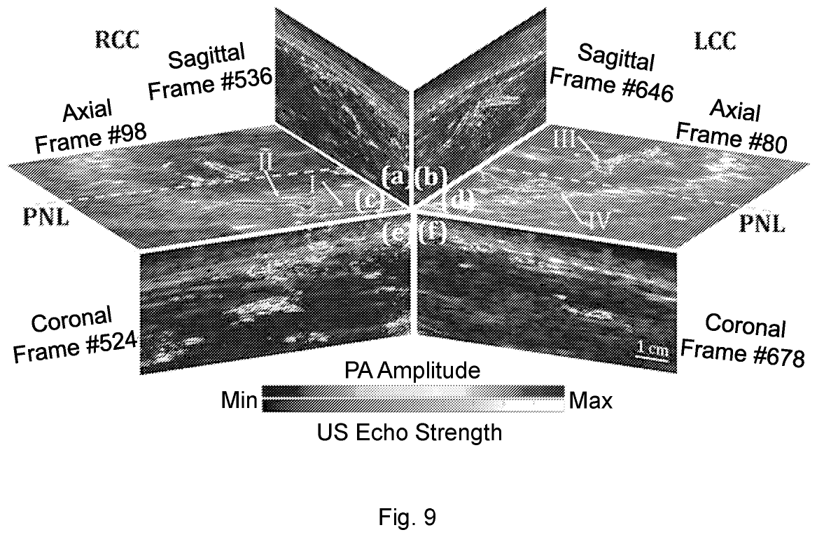

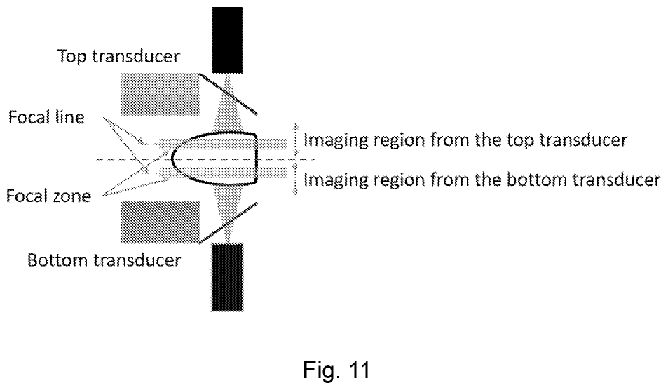

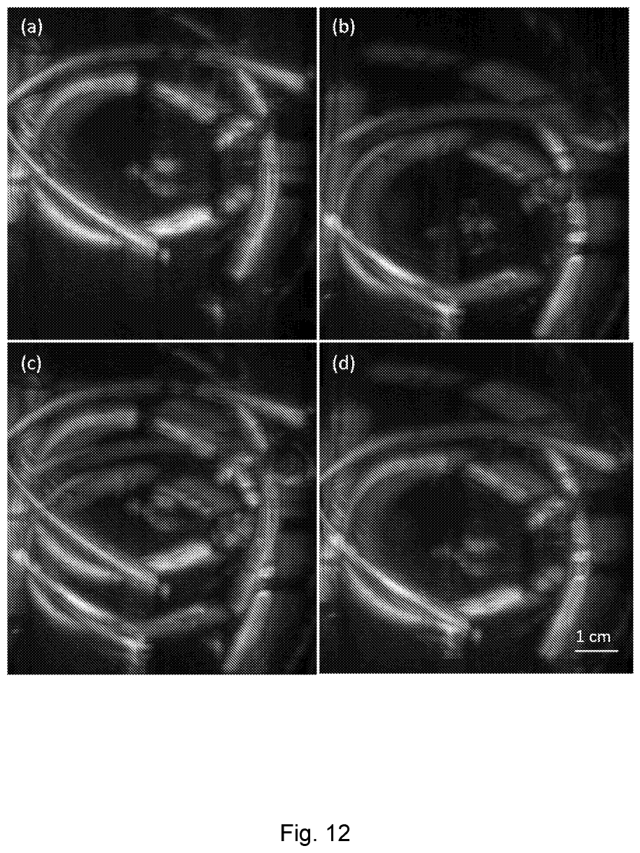

To combine the reconstructed data from two transducers, the offsets were identified in three dimensions. For alignment in the horizontal plane, the bottom transducer was used to reconstruct the top breast surface and cross-correlated the reconstructed MIP image with the one obtained from the top transducer for the same surface. To further verify the offset, the bottom breast surface was also reconstructed from two transducers and data was cross-correlated in the same manner. Calibration along the axial directions of the two transducers was achieved by lining up the same breast surface in the two reconstructed images. Once all the offsets are identified, they were compensated by shifting data along representative directions and then combined the two datasets. For overlapping regions in the 3D data, in order to achieve improved spatial resolution, for each transducer we chose the imaging region closest to the transducer's focal zone (as shown in FIG. 11), and then combined both. After combination, the 3D data was color-encoded and presented in a top-down view for better comparison with mammography images. Sample images regarding transducer alignment are shown in FIG. 9.

### Results

### A. System Resolution

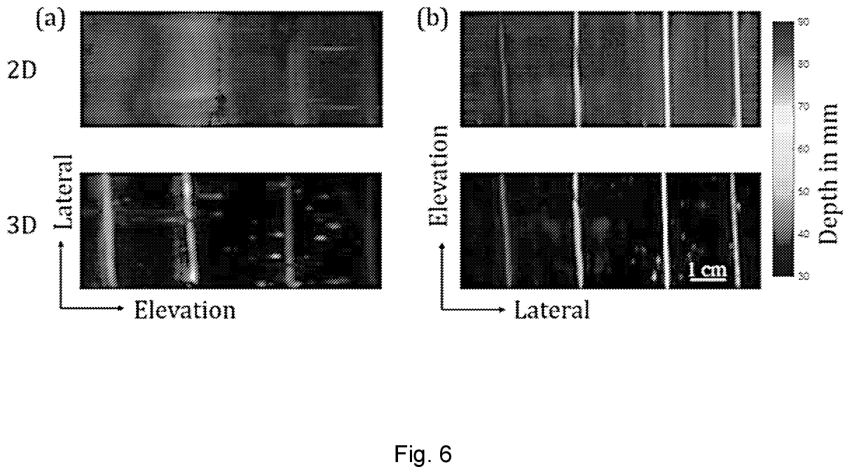

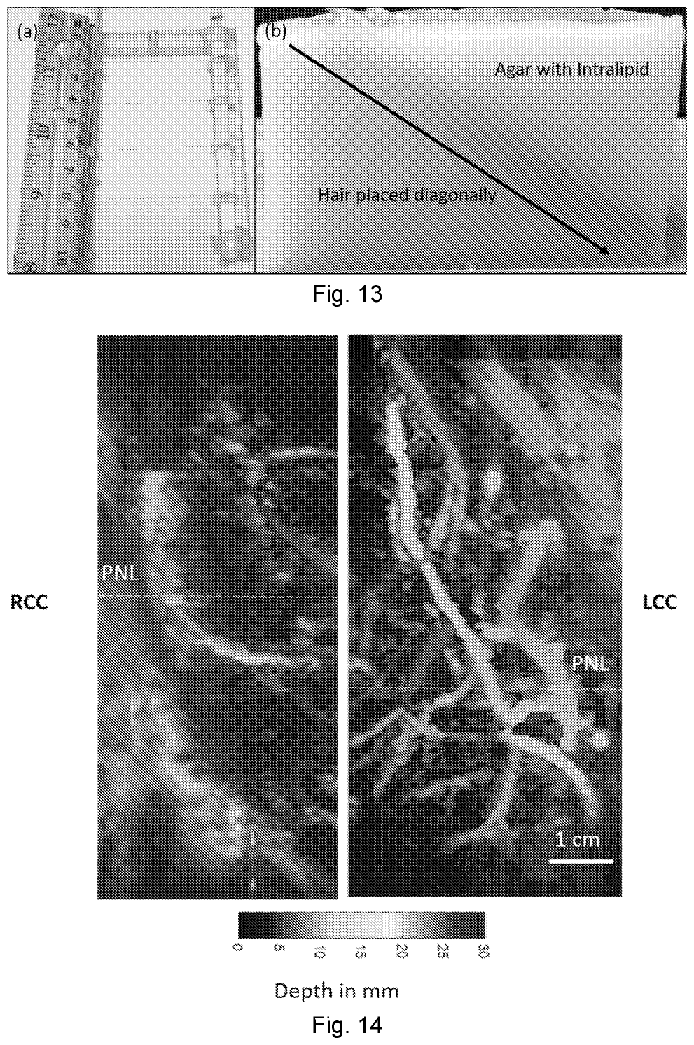

Initially, the system was tested using breast-like phantoms to experimentally quantify the resolution. We imaged an agar phantom of 60 mm height in 1% concentration of Intralipid 20% (IL-20) with four strands of black human hair embedded at different depths (FIG. 13 is a photo of the phantom). The phantom was scanned for 80 mm in both lateral and elevation directions to the transducer. Two methods were used for image reconstruction—(1) 2D Reconstruction, and (2) 3D focal-line reconstruction. Results are shown for lateral and elevation resolution in FIGS. 6(a) and 6(b) respectively. Elevation resolution is defined as the resolution along the axis orthogonal to the imaging plane of the transducer. It was computed using the full-width half maximum (FWHM) of the imaged hair. As shown in Table I, the average elevation resolution was 1.47 mm using 2D reconstruction and 1.05 mm using 3D reconstruction, respectively. Lateral resolution is the measure of spatial resolution along the length of the transducer and is defined by the element pitch of the transducer. Table II gives a detailed overview of lateral resolution for each hair strand.

TABLE I
Elevation Resolution

| Hair | Distance (mm) | 2D (mm) | 3D (mm) |
|---|---|---|---|
| 1 | 40 | 1.47 | 1.05 |
| 2 | 50 | 4.32 | 1.54 |
| 3 | 60 | 6.51 | 1.85 |
| 4 | 70 | 7.54 | 2.13 |

TABLE II
Lateral Resolution

| Hair | Distance (mm) | 2D (mm) | 3D (mm) |
|---|---|---|---|
| 1 | 50 | 1.07 | 1.05 |
| 2 | 60 | 0.97 | 0.84 |
| 3 | 70 | 0.95 | 0.91 |
| 4 | 80 | 1.17 | 1.08 |

### B. In Vivo Imaging Results

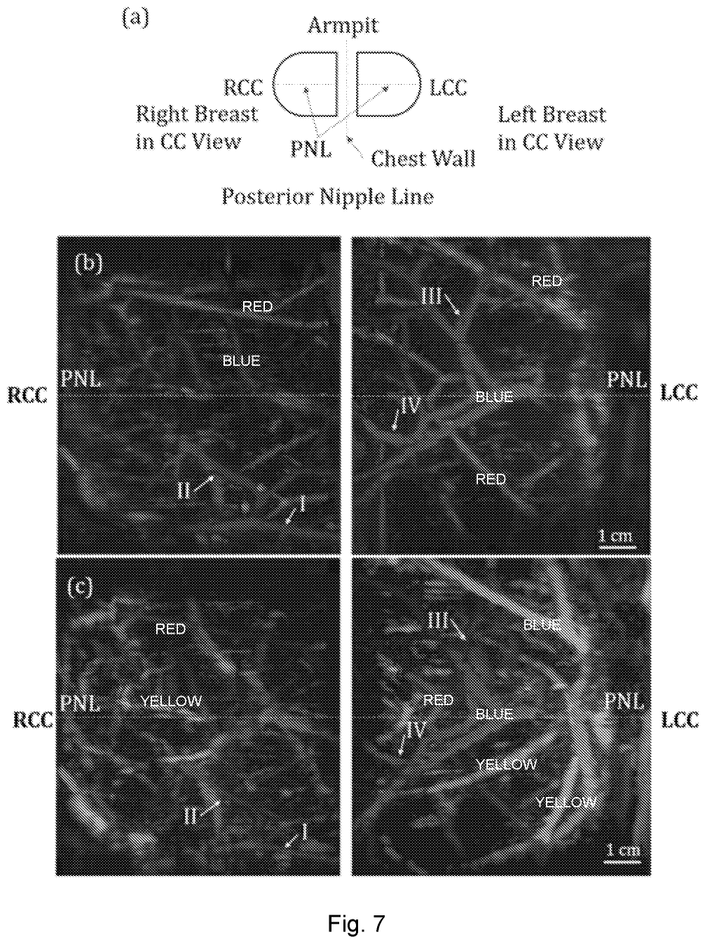

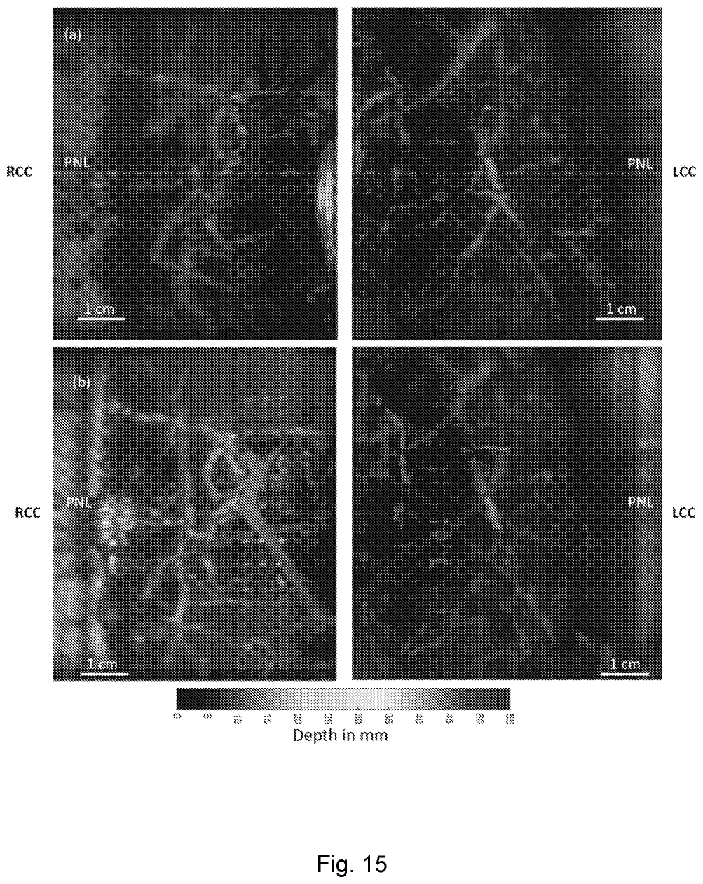

In order to test the exemplary system characteristics and usability, a 54-year-old volunteer with a breast cup size of D and scattered fibroglandular breast density was imaged. Length and thickness of the breast after compression were approximately 8 cm and 7 cm, respectively. Therefore, scan time for each breast was 80 seconds (0.1 mm step size at 10 Hz pulse repetition frequency). Total imaging time from start to finish was about 15 minutes for both breasts together. The raw-channel data was then processed to form a depth-encoded MIP image (processing details can be found in the Methods section). FIG. 7(a) shows how the images were represented in the orientation of a mammogram. FIGS. 7(b) and 7(c) show the 2D and 3D reconstructed depth-encoded MIP for the complete 7 cm of breast, respectively. The color scale from blue to red represents shallow to deep. Therefore, the vessels in blue are closest to the top skin surface, while the ones in red are the closest to the bottom surface of the breast. From the two images, it is obvious that the 3D focal-line reconstruction algorithm greatly improved the image quality at deeper depths. Vessels near the middle plane of the breast (with yellow color) can hardly be seen in the 2D reconstructed image, while they are clearly displayed in the 3D reconstructed image. To the best of our knowledge, this is the first time that a photoacoustic system provided imaging through 7 cm of breast tissue in human. Additionally, unlike systems compressing breast against the chest wall, no breathing artifacts were noted using the DSM system. As over 99% of women in the US have cup size of DD or smaller, volunteers of cup sizes B (average breast size in the US), D, and DD were imaged. Results for B and DD are presented in the figures. All human procedures were performed in compliance with University at Buffalo IRB protocol. All volunteers were enrolled after consent documents were signed. The imaging results are provided in the FIGS. 14 and 15.

C. Comparison with Contrast Enhanced MRI

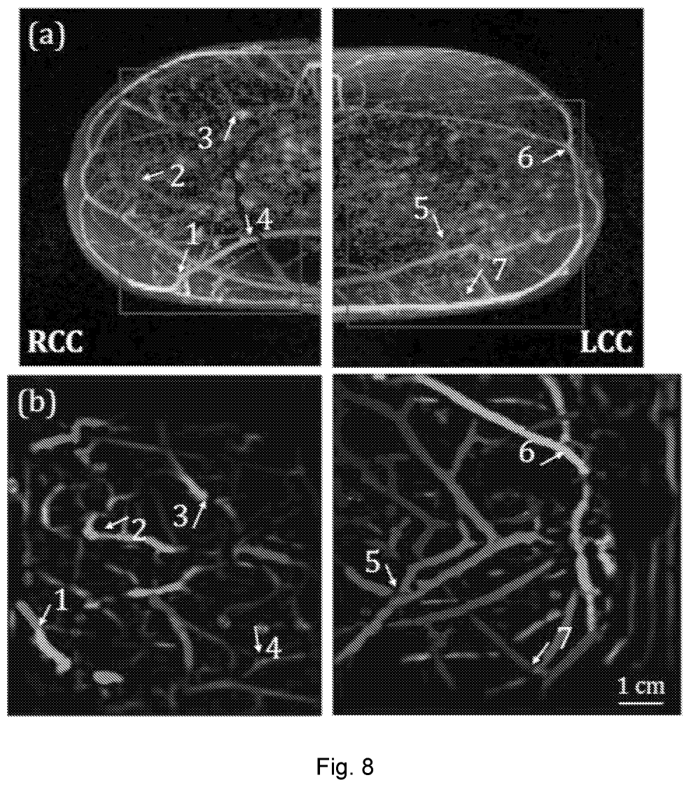

To further verify vascular images obtained using experimental embodiments, the data were compared with contrast-enhanced MRI subtraction images of pre-contrast injection and 6 minutes' post-contrast injection, as these images should highlight the vessels in the breast. For easy comparison, the same color encoding was performed for the MRI frames in FIG. 8, the same vessels are marked with numbers on the MRI and PA images. To enhance the vascular features, Frangi vessel filtering was performed on the PA data. Due to differences in compression between the present imaging procedure and MRI, there are slight differences in vascular structures. However, the same vessels were able to be identified without performing any image deformation, unlike other systems that compress breasts towards the chest wall. It should be noted that PA shows more vascular structures than MRI (FIGS. 8(a) and 8(b)), because PA is highly sensitive to the hemoglobin distribution. Furthermore, PA does not require any exogenous contrasts to highlight vascular structures. By simply studying hemoglobin signal intensity in a mass for angiogenesis, we can potentially differentiate benign and malignant ones. By using a transducer array with more elements, we can get a full-view image of the breast as shown in MRI.

### D. Simultaneous Ultrasound and PA Imaging

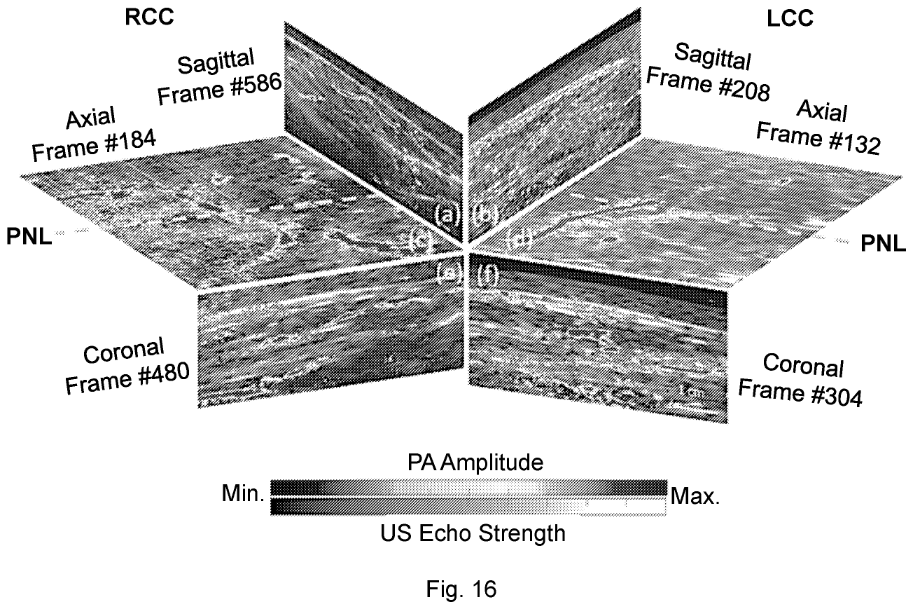

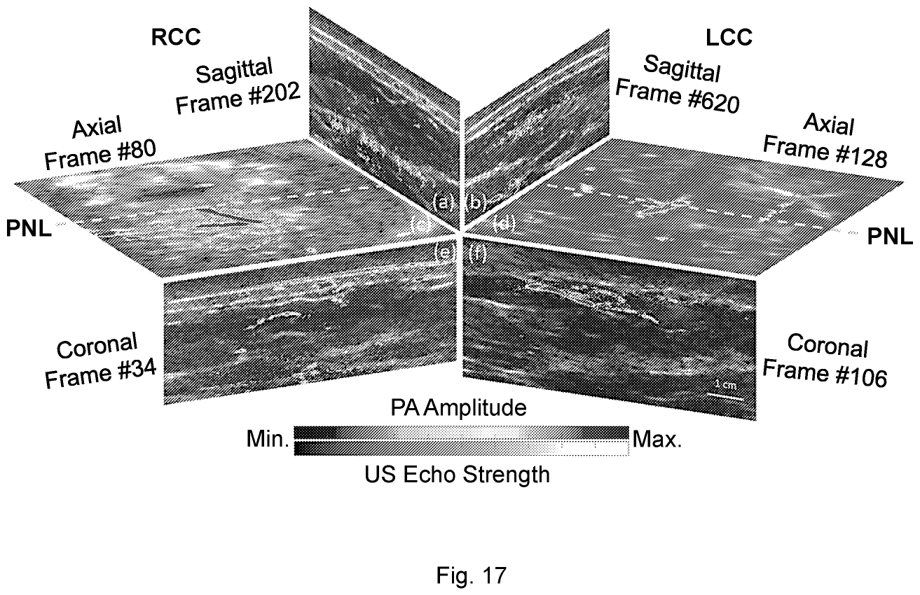

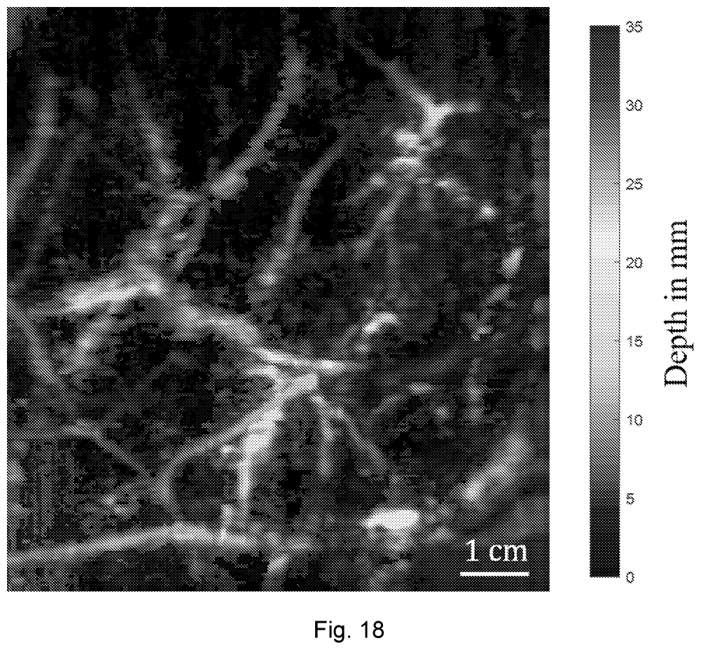

As mentioned in the Methods section, some embodiments of the presently-disclosed system can be configured to acquire both photoacoustic and ultrasound data. The two images are automatically co-registered because acquisition is by the same transducer array. To demonstrate the location of PA features in ultrasound, we overlaid the ultrasound images with photoacoustic images. Since ultrasound penetration depth can be much larger than that of photoacoustics, the ultrasound image from only one transducer was used. The same vascular structures in FIGS. 7 and 9, are marked by the same Roman numerals. FIGS. 9(a) and 9(b) are sagittal views, which is the conventional view of an ultrasound transducer. The particular slice selected from the right and left breasts are at 5.36 cm and 6.46 cm from the chest wall, respectively. On the other hand, FIGS. 9(c) and 9(d) are axial views sliced at different elevation depths for the right breast and left breast, at 0.80 cm and 0.98 cm from the top breast surface respectively. FIG. 9(e) is the coronal view of the right breast, 0.94 cm from the Posterior Nipple Line (PNL) and FIG. 9(f) is 2.48 cm from the PNL of the left breast. Our unique setup allows for clear visualization of the photoacoustic vascular contrast on top of ultrasonic soft tissue contrast. The combined photoacoustic and ultrasound results for Volunteers of cup size B and DD are shown in the FIGS. 16 and 17, respectively.

### Discussion

We have developed a mild compression photoacoustic system with high spatial resolution, fast imaging capability, and convenient correlation with all imaging modalities used for breast cancer detection—mammography, MRI, and ultrasound. The upright positioning allows for easy and quick patient imaging. Having light illumination from both sides of the breast stems the exponential decay of signal inside tissue to an extent, making it possible to get better images. Acquiring photoacoustic data from two transducers and combining it bolsters the signal intensity at the deepest portion of the breast where there is maximum attenuation. This maximizes our chances to see deep into the breast without missing any vascular features. After reconstruction and combination of images from top and bottom, we could see completely through 7 cm of breast tissue. With high resolution demonstrated by the DSM using the 3D focal-line reconstruction, we have the capability to detect small tumors in the sub-millimeter range, provided they exhibit sufficiently developed angiogenesis. Since the DSM acquires ultrasound simultaneously, it can obtain a comprehensive analysis of the breast by overlaying PA images on ultrasound.

Compared to existing photoacoustic breast imaging systems, the present approach is unique and advantageous in several aspects. Firstly, the DSM uses compression in the CC plane, similar to an X-ray mammogram, unlike other systems that use breast compression towards chest wall. Our compression geometry presents data in a view that radiologists are most familiar with and it facilitates correlation of PAT features with mammogram and MRI. Secondly, in the present system, we use ultrasound gel as a coupling medium, whereas other systems use water that needs to be changed out after every patient, which slows down their imaging time. In addition, as the breast is fully immersed in a coupling medium in other systems, future translation to PA-guided biopsy is almost impossible. Thirdly, the DSM is portable and can quite conveniently fit into small clinical spaces. In comparison, other systems have their patient lying in prone position and the breast pendant through an opening in a patient bed that is stationary. These systems take up more clinical space and are immovable. Lastly, unlike the dedicated PA mammogram systems that can only acquire photoacoustic images, the DSM also acquires ultrasound data using the same transducer, along with PA acquisition. The automatically co-registered ultrasound and photoacoustic images offer comprehensive information of breast tissue. To the best of our knowledge, there exists no other photoacoustic breast imaging system that has the above-mentioned advantages.

Low sensitivity to dense breast tissue has been a long-standing issue for X-ray mammogram. While ultrasound is unaffected by tissue density, it offers limited breast characterization. The DSM technique proposed in this study acquires both ultrasonic and photoacoustic features of breast and can potentially complement mammogram in screening procedures. Furthermore, images obtained from DSM can be directly compared to both mammogram and MRI, because data is acquired and presented in the CC plane, a format radiologists are most familiar with. For the patient, mild compression, fast imaging time, lack of ionizing radiation, and the noninvasive nature of this technique make it very comfortable as opposed to other imaging techniques. For the operator, the portable DSM system can be effortlessly stored and the acquisition procedure is similar to a mammogram. Therefore, incorporating this modality into breast cancer screening procedures would be fairly straightforward. Moreover, since DSM obtains vascular characteristics using optical contrasts, it has high potential to reduce unnecessary biopsies. As the first portable and upright photoacoustic mammography system, we believe that DSM will play a significant role in the field of breast cancer screening.

In the following, various further examples of the present disclosure are described:

### EXAMPLE 1

A system for imaging a sample, comprising: a pulsed light source configured to irradiate a region of interest in a sample from a first side and a second side opposite the first side; a first ultrasound transducer configured to receive acoustic waves induced at the region of interest and received from the first side of the sample; a second ultrasound transducer configured to receive acoustic waves induced at the region of interest and received from the second side of the sample; and a controller in electronic communication with the light source, the first ultrasound transducer, and the second ultrasound transducer, and wherein the controller is configured to: trigger a first light pulse from the light source; receive a first acoustic wave signal from the first ultrasound transducer, the first acoustic wave signal corresponding to the first light pulse; receive a second acoustic wave signal from the second ultrasound transducer, the second acoustic wave signal corresponding to the first light pulse; and construct an image of the region of interest based on the received first acoustic wave signal and second acoustic wave signal.

### EXAMPLE 2

The system of Example 1, wherein the controller is further configured to: trigger a first acoustic pulse from the first ultrasound transducer; and receive a first echo signal from the first ultrasound transducer, the first echo signal corresponding to the first acoustic pulse.

### EXAMPLE 3

The system of Example 2, wherein the controller is further configured to: trigger a second acoustic pulse from the second ultrasound transducer; receive a second echo signal from the second ultrasound transducer, the second echo signal corresponding to the second acoustic pulse; and wherein the second acoustic pulse is timed so as not to interfere with the first acoustic pulse and first echo signal.

### EXAMPLE 4

The system of any one of Examples 1-3, further comprising a fiber optic bundle configured to receive light from the light source and transmit the received light to the first side of the sample and/or the second side of the sample.

### EXAMPLE 5

The system of any one of Examples 1-4, further comprising: a first mirror configured to cause light from the light source at the first side to be coincident with the acoustic wave received at the first ultrasound transducer; and a second mirror configured to cause light from the light source at the second side to be coincident with the acoustic wave received at the second ultrasound transducer.

### EXAMPLE 6

The system of Example 5, wherein the first mirror and/or the second mirror is a dichroic mirror or acoustic mirror.

### EXAMPLE 7

The system of any one of Examples 1-6, further comprising: a first vessel configured to hold a medium on the first side of the sample, the first vessel having a first sample surface configured to be coupled to the first side of the sample, and wherein the first ultrasound transducer is contained within the first vessel such that acoustic waves from the sample are received by transmission through the first sample surface; and a second vessel configured to hold a medium on the second side of the sample, the second vessel having a second sample surface configured to be coupled to the second side of the sample, and wherein the second ultrasound transducer is contained within the second vessel such that acoustic waves from the sample are received by transmission through the second sample surface.

### EXAMPLE 8

The system of Example 7, wherein the first sample surface and the second sample surface are made from a material that is transparent to acoustic waves and light.

### EXAMPLE 9

The system of Example 8, wherein the first and second sample surfaces are each made from a fluorinated ethylene propylene (FEP) plastic film having a thickness of 50 μm or less.

### EXAMPLE 10

The system of Example 7, wherein the first vessel or the second vessel is mounted to a translatable stage configured to translate the first and second vessels closer to, or further from, each other, thereby compressing or uncompressing the sample, respectively.

### EXAMPLE 11

The system of Example 7, wherein the first and second vessels contain de-ionized water.

### EXAMPLE 12

The system of any one of Examples 1-11, wherein the light source is configured to emit light in the near-infrared (NIR) region.

### EXAMPLE 13

The system of any one of Examples 1-12, wherein the light source is a laser.

### EXAMPLE 14

The system of any one of Examples 1-13, wherein each of the first ultrasound transducer and the second ultrasound transducer is a linear array of ultrasound elements.

### EXAMPLE 15

The system of any one of Examples 1-14, further comprising a scan stage configured to translate the light at the first side and the light at the second side in a scan direction so as to impinge on a second region of interest of the sample, and to translate the first and second ultrasound transducers so as to receive acoustics waves from the second region of interest, and wherein the controller is further configured to actuate the scan stage to the second region of interest and repeat the trigger, receive, and construct steps at the second region of interest.

### EXAMPLE 16

The system of Example 15, wherein the controller is further configured to repeat the translate, trigger, and receive steps so as to construct an image of the sample over a distance in the scan direction.

### EXAMPLE 17

The system of any one of Examples 1-16, wherein the controller is further configured to perform ultrasound tomography.

### EXAMPLE 18

A method for imaging a sample, comprising: triggering a first light pulse from a light source configured to irradiate a region of interest in the sample from a first side of the sample and/or a second side of the sample opposite the first side; receiving, from the first side of the sample, a first acoustic wave signal induced at the region of interest by the first pulse; receiving, from the second side of the sample, a second acoustic wave signal induced at the region of interest by the first pulse; and constructing an image of the region of interest based on the received first acoustic wave signal and second acoustic wave signal.

### EXAMPLE 19

The method of Example 18, further comprising: triggering a first acoustic pulse configured to impinge upon the region of interest from the first side of the sample; and receiving, from the first side of the sample, a first echo signal induced by the first acoustic pulse.

### EXAMPLE 20

The method of Example 19, further comprising: triggering a second acoustic pulse configured to impinge upon the region of interest from the second side of the sample, and wherein the second acoustic pulse is timed so as not to interfere with the first acoustic pulse and first echo signal; and receiving, from the second side of the sample, a second echo signal induced by the second acoustic pulse.

### EXAMPLE 21

The method of any one of Examples 18-20, further comprising repeating the steps of triggering, receiving, and constructing at a second region of interest of the compressed sample.

### EXAMPLE 22

The method of any one of Examples 18-21, further comprising compressing at least a portion of the sample, wherein the sample is compressed along an axis from the first side to the second side.

Although the present disclosure has been described with respect to one or more particular embodiments, it will be understood that other embodiments of the present disclosure may be made without departing from the spirit and scope of the present disclosure.

What is claimed is:

1. A system for imaging a sample, comprising:
a pulsed light source configured to irradiate a region of interest in a sample from a first side and a second side opposite the first side;
a first ultrasound transducer configured to receive acoustic waves induced at the region of interest and received from the first side of the sample;
a second ultrasound transducer configured to receive acoustic waves induced at the region of interest and received from the second side of the sample; and
a controller in electronic communication with the light source, the first ultrasound transducer, and the second ultrasound transducer, and wherein the controller is configured to:
trigger a first light pulse from the light source;
receive a first acoustic wave signal from the first ultrasound transducer, the first acoustic wave signal corresponding to the first light pulse;
receive a second acoustic wave signal from the second ultrasound transducer, the second acoustic wave signal corresponding to the first light pulse; and
construct an image of the region of interest based on the received first acoustic wave signal and second acoustic wave signal.

2. The system of claim 1, wherein the controller is further configured to:
trigger a first acoustic pulse from the first ultrasound transducer; and
receive a first echo signal from the first ultrasound transducer, the first echo signal corresponding to the first acoustic pulse.

3. The system of claim 2, wherein the controller is further configured to:
trigger a second acoustic pulse from the second ultrasound transducer;
receive a second echo signal from the second ultrasound transducer, the second echo signal corresponding to the second acoustic pulse; and
wherein the second acoustic pulse is timed so as not to interfere with the first acoustic pulse and first echo signal.

4. The system of claim 1, further comprising a fiber optic bundle configured to receive light from the light source and transmit the received light to the first side of the sample and/or the second side of the sample.

5. The system of claim 1, further comprising:
a first mirror configured to cause light from the light source at the first side to be coincident with the acoustic wave received at the first ultrasound transducer; and
a second mirror configured to cause light from the light source at the second side to be coincident with the acoustic wave received at the second ultrasound transducer.

6. The system of claim 5, wherein the first mirror and/or the second mirror is a dichroic mirror or acoustic mirror.

7. The system of claim 1, further comprising:
a first vessel configured to hold a medium on the first side of the sample, the first vessel having a first sample surface configured to be coupled to the first side of the sample, and wherein the first ultrasound transducer is contained within the first vessel such that acoustic waves from the sample are received by transmission through the first sample surface; and
a second vessel configured to hold a medium on the second side of the sample, the second vessel having a second sample surface configured to be coupled to the second side of the sample, and wherein the second ultrasound transducer is contained within the second vessel such that acoustic waves from the sample are received by transmission through the second sample surface.

8. The system of claim 7, wherein the first sample surface and the second sample surface are made from a material that is transparent to acoustic waves and light.

9. The system of claim 8, wherein the first and second sample surfaces are each made from a fluorinated ethylene propylene (FEP) plastic film having a thickness of 50 μm or less.

10. The system of claim 7, wherein the first vessel or the second vessel is mounted to a translatable stage configured to translate the first and second vessels closer to, or further from, each other, thereby compressing or uncompressing the sample, respectively.

11. The system of claim 7, wherein the first and second vessels contain de-ionized water.

12. The system of claim 1, wherein the light source is configured to emit light in the near-infrared (NIR) region.

13. The system of claim 1, wherein the light source is a laser.

14. The system of claim 1, wherein each of the first ultrasound transducer and the second ultrasound transducer is a linear array of ultrasound elements.

15. The system of claim 1, further comprising a scan stage configured to translate the light at the first side and the light at the second side in a scan direction so as to impinge on a second region of interest of the sample, and to translate the first and second ultrasound transducers so as to receive acoustics waves from the second region of interest, and wherein the controller is further configured to actuate the scan stage to the second region of interest and repeat the trigger, receive, and construct steps at the second region of interest.

16. The system of claim 15, wherein the controller is further configured to repeat the translate, trigger, and receive steps so as to construct an image of the sample over a distance in the scan direction.

17. The system of claim 1, wherein the controller is further configured to perform ultrasound tomography.

18. A method for imaging a sample, comprising:
triggering a first light pulse from a light source configured to irradiate a region of interest in the sample from a first side of the sample and/or a second side of the sample opposite the first side;
receiving, from the first side of the sample, a first acoustic wave signal induced at the region of interest by the first pulse;
receiving, from the second side of the sample, a second acoustic wave signal induced at the region of interest by the first pulse; and
constructing an image of the region of interest based on the received first acoustic wave signal and second acoustic wave signal.

19. The method of claim 18, further comprising:
triggering a first acoustic pulse configured to impinge upon the region of interest from the first side of the sample; and
receiving, from the first side of the sample, a first echo signal induced by the first acoustic pulse.

20. The method of claim 19, further comprising:
triggering a second acoustic pulse configured to impinge upon the region of interest from the second side of the sample, and wherein the second acoustic pulse is timed so as not to interfere with the first acoustic pulse and first echo signal; and
receiving, from the second side of the sample, a second echo signal induced by the second acoustic pulse.

21. The method of claim 18, further comprising repeating the steps of triggering, receiving, and constructing at a second region of interest of the compressed sample.

22. The method of claim 18, further comprising compressing at least a portion of the sample, wherein the sample is compressed along an axis from the first side to the second side.
